(19) United States
(12) Patent Application Publication
(10) Pub. No.: US 2013/0172907 A1
(43) Pub. Date: Jul. 4, 2013

(54) SYSTEM AND METHOD FOR SPATIAL LOCATION AND TRACKING

(71) Applicant: Bernard Michael HARRIS, Kfar Saba (IL)

(72) Inventor: Bernard Michael HARRIS, Kfar Saba (IL)

(21) Appl. No.: 13/727,910

(22) Filed: Dec. 27, 2012

Related U.S. Application Data
(60) Provisional application No. 61/582,486, filed on Jan. 2, 2012.

(51) Int. Cl. A61B 19/00

(52) U.S. Cl. CPC A61B 19/201
USPC 606/130

(57) ABSTRACT

Method and system for navigated medical procedures includes a transmitter array having at least three ultrasound transmitters and at least one optical transmitter, and a receiver array having at least three ultrasound receivers and at least one optical receiver. In each transmission, the optical transmitter and only one ultrasound transmitter transmit signals. Distance measurements between each ultrasound transmitter and each ultrasound receiver are calculated based on time delays between reception of signals transmitted in each transmission and a speed of sound. A three-dimensional location of a transmitter relative to a receiver, or vice versa, is determined from at least three calculated distance measurements. Placement of the transmitter or receiver array on a surgical tool, a medical implant or instrument, or a part or point in or on a patient's body, or other object used in the medical procedure, enables the three-dimensional location thereof to be viewed on a display.

### CROSS REFERENCE TO RELATED APPLICATION

This application claims priority under 35 U.S.C. §119(e) of U.S. provisional patent application Ser. No. 61/582,486 filed Jan. 2, 2012, the entire disclosure of which is incorporated by reference herein.

### FIELD OF THE INVENTION

The present invention relates generally to three-dimensional location and tracking of objects, with particular application to spatial tracking of surgical tools and implants in medical procedures, such as computer aided surgery. The present invention also provides an improved method, system and arrangement for determining the location and/or orientation of medical instruments and anatomical features.

### BACKGROUND OF THE INVENTION

Surgical navigation which is also referred to as computer aided surgery or guided surgery, is a well-established technique to aid surgeons in three-dimensional (3D) locating of instruments, implants and prostheses relative to a patient's body. For example, precision guidance of instruments during neurosurgery is critical in minimizing the impact on brain tissue. Similarly, correct positioning of the acetabular cup during total hip arthroplasty (THA) diminishes the risk of complications leading to revision surgery. A closely related application to THA is Total Knee Arthroplasty (TKA).

In the context of THA and TKA, important attributes such as implant angles and locations are presented to the surgeon during the operation on a display so that they can orient implants optimally before fixing them in place on the patient's bones.

One well-established method of measuring distance and calculating position utilizes a time delay between an optical or electromagnetic signal and an acoustic signal. In this regard, U.S. Pat. No. 4,751,689 to Kobayashi describes a single channel system using radio waves, U.S. Pat. No. 4,207,571 to Passey describes a navigational system with multiple receivers, U.S. Pat. No. 4,814,552 to Stefik et al. describes a method for an input device such as a stylus, U.S. Pat. No. 5,191,328 to Nelson describes using this method for hitching a trailer. U.S. Pat. Nos. 5,920,395 to Schultz, 5,197,476 to Nowacki, 5,617,857 to Chader, 5,848,967 to Cosman, as well as numerous others, all describe methods for providing spatial information during medical procedures. U.S. Pat. No. 5,230,623 to Guthrie et al. describes a three-dimensional location system for medical operations which claims the use of triangulation of time delays using ultrasonic senders or receivers. The time of flight of an ultrasonic signal from sources on a pointer or medical tool to two or more receivers is used to estimate the orientation of the pointer in space.

Various methods for computer aided medical procedures requiring spatial information have also been proposed, with the method of using stereo cameras and passive or active markers in common commercial use. One such method is described by Nowacki and by Guthrie, and systems in commercial use are more fully described in U.S. Pat. Nos. 5,880,976 and 6,205,411 to DiGioia. Commercial systems, include but are not limited to the Brainlab Kolibri and VectorVision systems (Brainlab AG, Feldkirchen Germany) and Aesculap Orthopilot system (Aesculap AG, Tutlingen, Germany), Stryker Navigation (Kalamazoo, Mich., USA) and others. In these systems, a stereoscopic camera tracks passive arrays of reflective spheres or active arrays of LEDs to determine the location and orientation of markers to which they are attached. The markers may be hand-held to indicate anatomical features, affixed to bone or connected to surgical tools.

Similar to the triangulation method of Guthrie, U.S. Pat. No. 8,000,926 to Roche et al. uses a phase difference between a first sequence of ultrasonic signals and a second sequence of ultrasonic signals to estimate a difference between an expected location and an estimated location of a marker.

None of the above-referenced patents discloses a method for calculating a delay between optical and acoustic signals to provide spatial measurements during medical procedures.

### OBJECTS AND SUMMARY OF THE INVENTION

An object of the present invention is to provide an improved three-dimensional object locating and tracking system and method, with particular application to spatial tracking of surgical tools and implants in medical procedures, such as computer aided surgery.

Another object of the present invention is to provide an improved method, system and arrangement for determining the location and/or orientation of medical instruments and anatomical features.

A method for navigated medical procedures in accordance with the invention includes placing at least one transmitter array and at least one receiver array on at least one object used in the medical procedure, each transmitter array having at least three first transmitters and at least one second transmitter that transmits at a higher speed that the first transmitters and each receiver array including at least three first receivers that receive transmissions from the first transmitters and at least one second receiver that receives transmissions from the second transmitter. For example, the first transmitters and receivers may use ultrasound while the second transmitter and receiver use optics or infrared. The object may be a surgical tool, medical implant, medical instrument, or part or point in or on the patient's body.

A sequence of transmissions is scheduled by a control unit for the transmitters, wherein in each transmission, the second transmitter and only one of the first transmitters transmit signals and the respective transmitted signals are received by the second receiver and all of the first receivers. Using a measurement system, distance measurements between each first transmitter and each first receiver are calculated based on time delays between reception of the signals transmitted in each transmission and a speed of sound. A three-dimensional location of at least one of the first transmitters relative to the first receivers is determined from at least three calculated distance measurements between the first transmitter and the first receivers, to thereby provide the three-dimensional location of the object(s). The three-dimensional location of each object to display information regarding the medical procedure.

A system for navigated medical procedures in accordance with the invention includes at least one transmitter array each having at least three first transmitters and at least one second transmitter that transmits at a higher speed that the first transmitters, and at least one receiver array, each having at least three first receivers that receive transmissions from the first transmitters and at least one second receiver that receives transmissions from the second transmitter. A measurement system controls the transmitter array and the receiver array to cause a sequence of transmissions by the transmitters, as described above, which are received by the receivers. The measurement system calculates distance measurements between each first transmitters and each first receivers based on time delays between reception of the signals transmitted in each transmission and a speed of sound. The measurement system also determines a three-dimensional location of at least one of the first transmitters relative to the first receivers from at least three calculated distance measurements between the first transmitter and the first receivers. Placement of the transmitter array or receiver array on at least one object used in the medical procedure enables the three-dimensional location of the at least one object to be viewed on the display.

### BRIEF DESCRIPTION OF THE DRAWINGS

The invention may best be understood by reference to the following detailed description of illustrative embodiments when read in conjunction with the accompanying drawings, wherein:

FIG. 1 provides a reference for an explanation of a principle of measurement used in the invention.

FIG. 2 is a block diagram of an exemplifying embodiment of a system in accordance with the invention.

FIG. 3 is a block diagram of an exemplifying embodiment of the electronics of a transmitter array in accordance with the invention.

FIG. 4 is a block diagram of an exemplifying embodiment of the electronics of a receiver array in accordance with the invention.

FIG. 5 shows an exemplifying embodiment of a transmitter array used as a marker in accordance with the invention.

FIG. 6 shows an exemplifying embodiment of a fixed array, with two receiver arrays and a compartment for holding a display, used in a system in accordance with the invention.

FIG. 7 shows an exemplifying embodiment of a complete system as used in a hip replacement operation in accordance with the invention.

FIG. 8 shows an exemplifying embodiment of a flow diagram for the sequence of events typically executed by a transmitter array transmitting a sequence of signals in response to a command from the receive array in accordance with the invention.

FIG. 9 shows an exemplifying embodiment of a flow diagram for the sequence of events typically executed by a receiver array sampling a sequence of signals transmitted in response to a command which it sent in accordance with the invention.

FIG. 10 shows an exemplifying embodiment of a temporary attachment to the patient's body using needles or similar sharp objects pushed against the bone rather than screwed into the bone used in a system in accordance with the invention.

FIG. 11 shows an exemplifying embodiment of guide temporarily inserted into the Acetabulum for improved registration of the Acetabular edge used in a system in accordance with the invention.

### DETAILED DESCRIPTION OF THE INVENTION

The description below refers to THA as an example of surgical navigation, but all of the described aspects of the invention are not limited to such surgery and can be applied to many medical and surgical situations without limitations, e.g., TKA as well as many variations of surgery for joint replacement or joint repair. Additional examples of applicable medical situations which can benefit from the invention and in which the invention may be applied include, but are not limited to, orthopaedic surgery, dentistry, spinal surgery, neurosurgery, ultrasound imaging, guided biopsy, guided delivery of radiation and any medical situation which requires two or three dimensional positioning, location and/or orientation of medical devices or equipment relative to a patient.

Generally, a system in accordance with the invention provides spatial information of objects to aid users during medical procedures. The underlying need is thus to measure distances between points on these items and to establish a spatial relationships between them. One fundamental measurement method is based on using a difference in the time of flight between an acoustic or ultrasound signal and an optical or electromagnetic signal to measure distance. This provides an improved and more accurate measurement than using ultrasound alone, and a compact and much lower cost solution than solutions based on image processing.

Accordingly, an objective of a system in accordance with the invention is to provide information to medical personnel on attributes of the location and/or orientation of objects or devices used during a medical procedure, including devices implanted into a patient's body during the procedure. These objects could be anatomical, for example, where the relevant attribute is the angles of the bones of the knee relative to each other, or could be medical devices such as a hip replacement implant, where the relevant attribute could be the angles between the implant and the pelvis. Similarly, the device could be a reamer and the attribute could be the location of the reamer indicative of the depth to which the reamer has drilled into bone, or the location of a handheld ultrasound scanner where the relevant attribute is the exact position and location of the scanner, so that multiple two-dimensional (2D) scans can be mathematically combined to provide a 3D ultrasound image.

As background of the operational principles of the invention, as the speed of light is far greater than the speed of sound, an ultrasound signal which is transmitted simultaneously with an optical signal will arrive at a receiver later than the optical signal. For example, if a LED which is co-located with an ultrasound transmitter is 1 meter from a photo-detector co-located with an ultrasound receiver, simultaneously transmitted optical and ultrasound signals will arrive, i.e., be received by the respective receivers, with a time delay between them of approximately 2.92 milliseconds. The optical signal therefore serves as a precise reference to measure the time that the acoustic signal takes to travel from transmitter to receiver. The terms acoustic and ultrasound are used interchangeably in this application. Any reference to ultrasound devices or signals is applicable to acoustic devices or signals, and vice versa. The terms optical and electromagnetic are used interchangeably in this application. Any reference to optical devices or signals is applicable to electromagnetic devices or signals, and vice versa. The terms optical and infrared are also used interchangeably in this application. Any reference to optical devices or signals is applicable to infrared devices or signals, and vice versa.

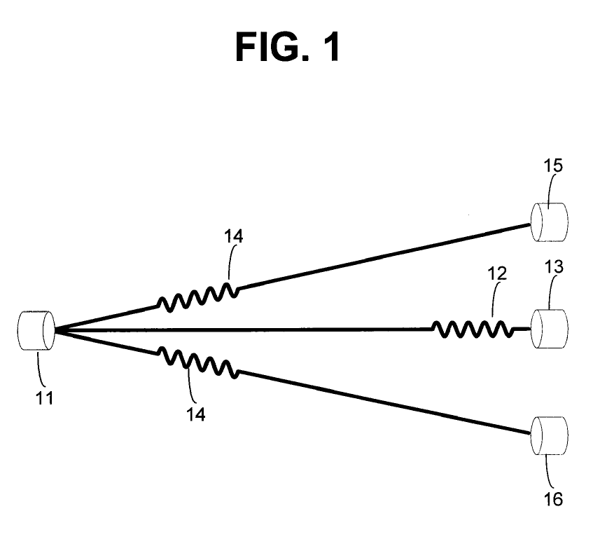

FIG. 1 illustrates the foregoing operational principle. Optical and acoustic signals 12, 14 are transmitted simultaneously from a transmitter 11. The optical signal 12 arrives at an optical receiver 13 almost instantaneously, while the acoustic signal 14 arrives at acoustic receivers 15 and 16 with a delay, i.e., there is a time difference between the time when the optical receiver 13 receives the optical signal 12 and the time when the acoustic receivers 15 receive the acoustic signal 14. The distance between the transmitter 11 and the receivers 15 and 16 can be calculated from the delay and the speed of sound.

With this principle in mind, measurement of the distance from a single transmitter to three non-colinear receivers allows calculation of the location of the transmitter by, for example, the mathematical method of trilateration. The distances R₁, R₂ and R₃ between (x,y,z), representing the three-dimensional location of the transmitter, and the points at P1=(x₁,y₁,z₁), P2=(x₂,y₂,z₂) and P3=(x₃,y₃,z₃), representing the three-dimensional location of the receivers are:

R₁²=(x−x₁)²+(y−y₁)²+(z−z₁)²

R₂²=(x−x₂)²+(y−y₂)²+(z−z₂)²

R₃²=(x−x₃)²+(y−y₃)²+(z−z₃)²

Formulas for the calculation of a point at coordinates Pₐ=(x,y,z) from three other points is given, for example, in a Wikipedia article on trilateration. Calculation of Pₐ is thus a straightforward mathematical operation of solving three unknowns (x,y,z) in three equations.

The distances R₁, R₂ and R₃ can be measured from the delays t1, t2 and t3 between the respective reception or arrival times of the ultrasound or acoustic signal at the ultrasound receivers, relative to the signal measured at the optical receiver nearby. (The term nearby is in the sense that the time for the optical signal to reach the optical receiver is so small that it can be ignored without causing appreciable error in calculations.)

R₁=ct₁;R₂=Ct₂;R₃=Ct₃;

where c is the speed of sound.

Knowledge of three non-colinear fixed points on a rigid body determines its orientation and location in a frame of reference. If three transmitters are located at fixed and known non-colinear positions Pₐ, P_{b} and P_{c} on a rigid body, the location and orientation of the body relative to three fixed receivers at P₁, P₂ and P₃ on a different body can be calculated from measurement of the distances between all of the receivers and all of the transmitters.

Thus, the location and orientation of two or more bodies relative to each other can be located in three-dimensional space using at least three acoustic transmitters or three acoustic receivers on each of the bodies, where an optical or electromagnetic signal serves as a reference to provide precise timing of the acoustic signals.

Ultrasound is frequently used in determining linear distances, typically by measuring the time for a reflected ultrasonic signal to return from a surface or from an interface between materials or tissues with different densities. This has limited accuracy and generally cannot provide the accuracy of approximately 1 mm needed in many surgical procedures. In this invention, the ultrasound signal of interest is not reflected but is transmitted from one point and received at another point, where the time of flight between the points provides the means to calculate the linear distance. There are two issues that constrain the accuracy of using ultrasound alone for measurement of distances, namely uncertainty of the speed of sound and the timing of the emitted ultrasonic signal. In the case of reflected signals, the transmitter and receiver are frequently co-located, and the timing of both the outgoing signal and the echo are measured by the same electronics. However, if the receivers are not aware of the exact time that the acoustic signal was transmitted, the distance between transmitter and receivers cannot be directly calculated, as the time of flight cannot be directly determined and only the time difference of arrival (tDOA) between multiple receivers can be used to calculate the location of the transmitter.

In cases where the distance between receivers is much smaller than the distance to the transmitter, the tDOA approach is generally less accurate than measuring the actual time of flight. This is because the receivers are closer to each other than to the transmitter and timing inaccuracies arise due to, for example, manufacturing tolerances or other dimensional errors. These inaccuracies are relatively smaller between a receiver and a distant transmitter than between two nearby receivers. As an example, receivers which are 100 mm apart and have uncertainty as to their precise location of 0.5 mm due to manufacturing tolerances, have an inaccuracy of 0.5% when using tDOA (0.5 mm/100 mm=0.5%). When using synchronized optical and ultrasound signals from a transmitter which is 50 cm away, the error would be only 0.1%, a factor of five improvement (0.5 mm/500 mm=0.1%).

In many situations where tDOA and multilateration are used to calculate spatial location, multiple receivers (five or more) are required to achieve a high level of accuracy, where the method of least squares estimation is frequently used. In addition, higher accuracy is achieved when the receivers are spaced widely apart, which is problematic in a surgical application as the components would need to be larger than is practical.

Another error source that can be improved is uncertainty regarding the speed of sound. The speed of sound is dependent on a number of factors, primarily temperature and humidity. The speed of sound is approximately 344 m/s at 20° C. and 50% relative humidity. Temperature causes a change of approximately 0.61 m/s for each degree change, and a 100% change in humidity causes an approximate change of 0.36% in the speed of sound at 20° C. Therefore, humidity and temperature cause uncertainty in calculating the distance from measurements of time delay. A formula for calculating the speed of sound as a function of temperature and humidity can be found in Cramer O., J. Acoust. Soc. Am. 93(5), 1993, p2510-2616; formula at p2514, incorporated by reference herein. An approximate formula for the speed of sound as a function of temperature c(T) in dry air is:

c(T)=331.3+0.606T (m/s)

where T is the temperature in degrees Celsius. This is approximately a 0.2% scaling factor for every degree Celsius. In a surgical environment where typical distances between items of interest are approximately 100 mm to 700 cm, the inaccuracy regarding the speed of sound translates to an inaccuracy of 0.2 mm to 1.4 mm per degree Celsius. In many cases, this is inadequate, as precision of better than 1 mm is desired.

If the exact speed of sound is unknown and a more precise calculation is desired, compensation for the uncertainty in the speed of sound can be accomplished in a number of ways:
Measurement of the air temperature at both the receivers and at the transmitters, and calculating the speed of sound from the average of the temperature dependent speed of sound at the two points.
Measurement of the air temperature and the humidity at both the receivers and at the transmitters, and calculating the speed of sound from the average of the temperature and humidity dependent speed of sound at the two points.
Measuring the actual speed of sound at either the transmitters or the receivers or at both, or on a separate device nearby which specifically measures the speed of sound. This can be done by having a receiver and a transmitter dedicated specifically for speed of sound measurement at a known fixed distance from each other and measuring the time for an acoustic signal to propagate between them.
Adding a fourth receiver (or in an equivalent manner, a fourth transmitter). The measured distances are related to the actual distances approximately linearly.

c_{estimated}=c_{actual}+Δc_{unknown}

Dᵢ=c_{estimated}tᵢ=c_{actual}tᵢ+Δc_{unknown}tᵢ

As Rᵢ=c_{actual}tᵢ the actual distance which needs to found

(l+k)·Dᵢ=Rᵢ(i=1,2,3)

where Dᵢ is the measured distance calculated from the measured tᵢ and calculated from the estimated speed of sound, Δc_{unknown} is the unknown difference between the actual and the measured speed of sound and k is a variable used to calculate the actual distances Rᵢ from the measured distances Dᵢ. By adding a fourth receiver at known position P₄=(x₄,y₄,z₄), the equations above become:

R₁²=(x−x₁)²+(y−y₁)²+(z−z₁)²=(l+k)²D₁²

R₂²=(x−x₂)²+(y−y₂)²+(z−z₂)²=(l+k)²D₁²

R₃²=(x−x₃)²+(y−y₃)²+(z−z₃)²=(l+k)²D₁²

R₄²=(x−x₄)²+(y−y₄)²+(z−z₄)²=(l+k)²D₁²

which comprises a set of four equations that can be solved for the four unknowns x,y,z and k. Equivalently adding a fourth transmitter at P_{d} at a known point on the same rigid body as Pₐ, P_{b} and P_{c} also results in a similar set of four equations in four unknowns which can be solved. It is straightforward to substitute for k to get the corrected distances Rᵢ.

Additional transmitters or receivers (more than four) allow for calculation of the distances Rᵢ where i>4 in a redundant manner. The system of equations may then be solved by making a least squares estimation of the coordinates (x,y,z) and the parameter k.

Another source of error which can be corrected for is manufacturing tolerances. Calibrating the transmitter or receivers in a controlled environment prior to use allows measurement of their precise positions on the rigid bodies. The deviation from the default positions are thus stored in memory and can be used to calculate for each particular manufactured part, rather than the default positions.

Similarly, variations in the measured time of flight due to the shape of the transmitters or receivers and the angle between them can be calculated and incorporated as correction factors in calculating the location and orientation of the bodies. For example if the sound source is located behind a tube or constriction within the transmitter, the sound will appear to come from the sound source when the receiver is directly in front of the transmitter, but will appear to come from the opening of the tube or the constriction when received from an angle. Compensating for angular variation can be accomplished by measurement and/or by geometrical calculations.

It is also possible to use a single optical or electromagnetic signal from a transmitter on one body and to measure the time difference of arrival at receivers on the other body. This is the same principle of operation of global positioning systems (GPS). However, the speed of light is such that high speed electronics are required to measure small time delays. For example, a difference of 1 cm between two receivers results in a time delay of only 1/30 nanosecond. Fortunately, electronic devices capable of measuring phase differences of less than 1° at 2 GHz are commonly available. This is equivalent to measurement of 1.4 picoseconds or less than 0.5 mm. An example of a phase detector with such capabilities is ON semiconductor MC100EP40, but multiple similar devices are commercially available.

In an optical solution, the output from two high speed photodetectors would feed into the phase detector to measure the phase difference. Such high speed detectors are commonly used in fiber optical communication equipment and would be simple to adapt to this application, and such adaption is considered to be within the scope of the invention. Measurement from four phase detectors, each connected to two of the receivers provides a solution based on time delay of arrival (tDOA), which can provide for the location of a source based on multilateration. Calculations can be found in the Wikipedia article on multilateration, incorporated by reference herein, or in references for the mathematics of GPS technology. Similarly, an RF signal could be picked up at two antennas and fed to a phase detector, instead of using an optical signal.

It is also possible to use multilateration only using acoustic signals, without any reference optical signal. The principles remain the same as in GPS or similar multilateration techniques, with the advantage of using low-cost and low speed components, but with the disadvantage of uncertainty in the precise speed of sound during the measurement. Using a reflected ultrasonic signal is also less accurate, as the reflected signal typically does not return from a single point and it is difficult to ascertain the exact point from which the echo returned without fairly sophisticated processing as in medical diagnostic ultrasound equipment. In spite of the disadvantage and lower accuracy, this embodiment is still considered to be within the scope of the invention.

Based on the gain in accuracy, the addition of a reference optical or electromagnetic signal is advantageous. Besides the improved accuracy, ultrasound components are often low cost and ready available and require simple electronics.

Although the description herein primarily mentions use of an optical signal from an optical transmitter to an optical receiver that travels at a speed greater than the speed of an ultrasound signal that travels from an ultrasound transmitter to an ultrasound receiver, any two different signals may be used in the invention between two sets of transmitters and receivers, provided one set operates with a higher speed wave or signal than the other and is thus received by the respective receiver before the slower speed wave or signal is received by the respective receivers. The invention thus does not require an optical signal for one set of transmitters and receivers, and ultrasound signals for the other set of transmitters and receivers. All that is required is for simultaneous transmission of different signals from a group of transmitters (including one transmitter transmitting waves or signals at one speed and a plurality of other transmitters transmitting waves or signals at a different, slower speed) so that the higher speed wave or signals is received at a respective receiver before the other signals are received at respective receivers (to thereby provide a reference for the later received signals). In this manner, a time delay is calculated based on the difference between the reception times and can be used, as developed elsewhere herein, to determine position of an array of the transmitter or an array of the receivers, or a rigid object to which the transmitter or receiver array is mounted.

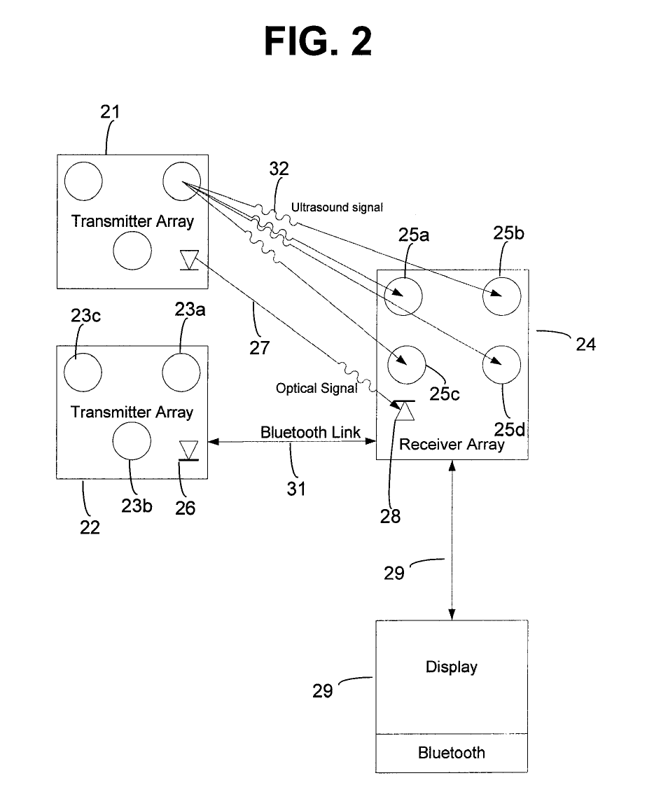

In the exemplary embodiment of FIG. 2, transmitter arrays 21 and 22 each have three ultrasound components such as ultrasound transmitters 23a, 23b, 23c, while a receiver array 24 has four ultrasound receivers 25a, 25b, 25c, 25d. Reference number 23 is used to refer to the ultrasound transmitting-capable component or transmitter generally and reference number 25 is used to refer to the ultrasound receiving-capable component or receiver generally. Different numbers of ultrasound transmitters in the transmitter arrays 21, 22 and different numbers of ultrasound receivers in the receiver array 24 are considered within the scope of the invention. The ultrasound receivers 25 are arranged in a non-co-linear arrangement. As used herein, transmission from a component may also be considered an emission from a component so that a transmitter may also be considered an emitter.

An infrared optical transmitter 26 on the transmitter array 21, 22 sends optical signals 27 to an optical receiver 28 on the receiver array 24. Similarly, each of the ultrasound transmitters 23a, 23b, 23c transmits acoustic signals 32 to the ultrasound receivers 25a, 25b, 25c, 25d. The receiver array 24 communicates with a display 29, for example, via a wireless link 30 such as Bluetooth, and also communicates with each of the transmitter arrays 21, 22 via a wireless link 31. Different communication techniques are considered within the scope of the invention and to this end, the transmitter arrays 21, 22, the receiver array 24 and the display 29 are each provided with an appropriate communications unit or capability to effect the desired communications technique(s).

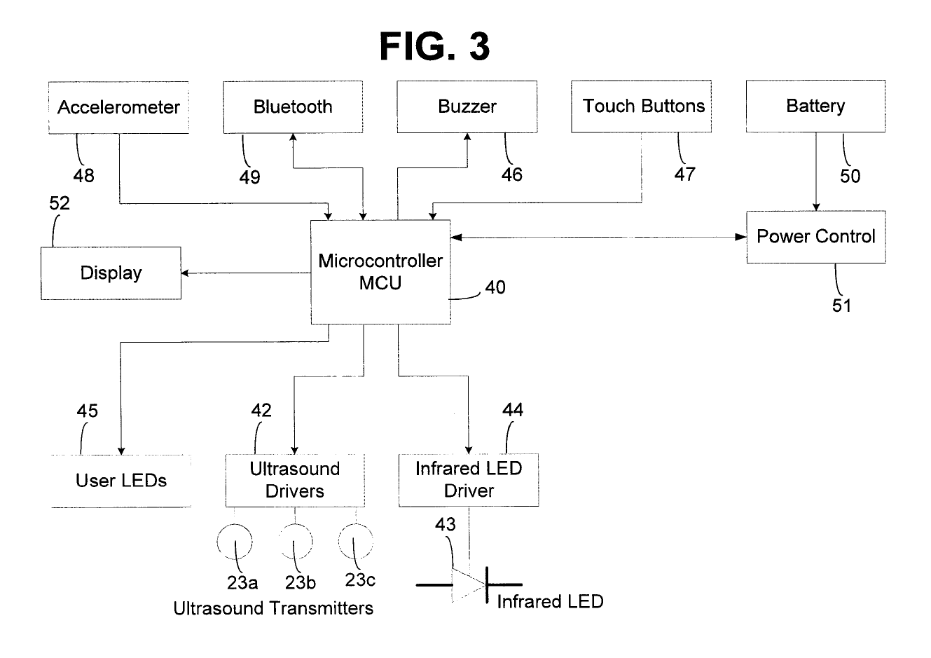

FIG. 3 is a block diagram of an exemplary embodiment of the electronics of a transmitter array 21, 22. The transmitter array 21, 22 is controlled by a microcontroller (MCU) 40, which has a number of functional blocks connected to it, each representing hardware and/or software to effect the described function(s) of the block. The ultrasound transmitters 23a, 23b, 23c are driven by one or more drivers 42 which provide the electrical energy at the appropriate frequency to the transmitters 23a, 23b, 23c under control by the MCU 40. Optical transmitter 26 comprises an infrared LED 43 driven by an infrared LED driver 44 which can simply be a transistor, and is coupled to the MCU 40 by any conventional electrical coupling means. User LEDs 45, a buzzer 46 and touch buttons 47, all of which are coupled to the MCU 40 by conventional electrical coupling means, provide a simple user interface. An optional three-axis accelerometer 48, also coupled to the MCU 40 by conventional coupling means, provides information on the orientation of the transmitter array 21, 22 relative to gravity. A Bluetooth module 49, also coupled to the MCU 40, provides a wireless communication link with the receiver array 24 and the display 29. A battery 50 and power controller 51, coupled to the MCU 40, provide the required voltages to the units on the array 21, 22. An optional integrated display 52, coupled to the MCU 40, presents visual information to the user.

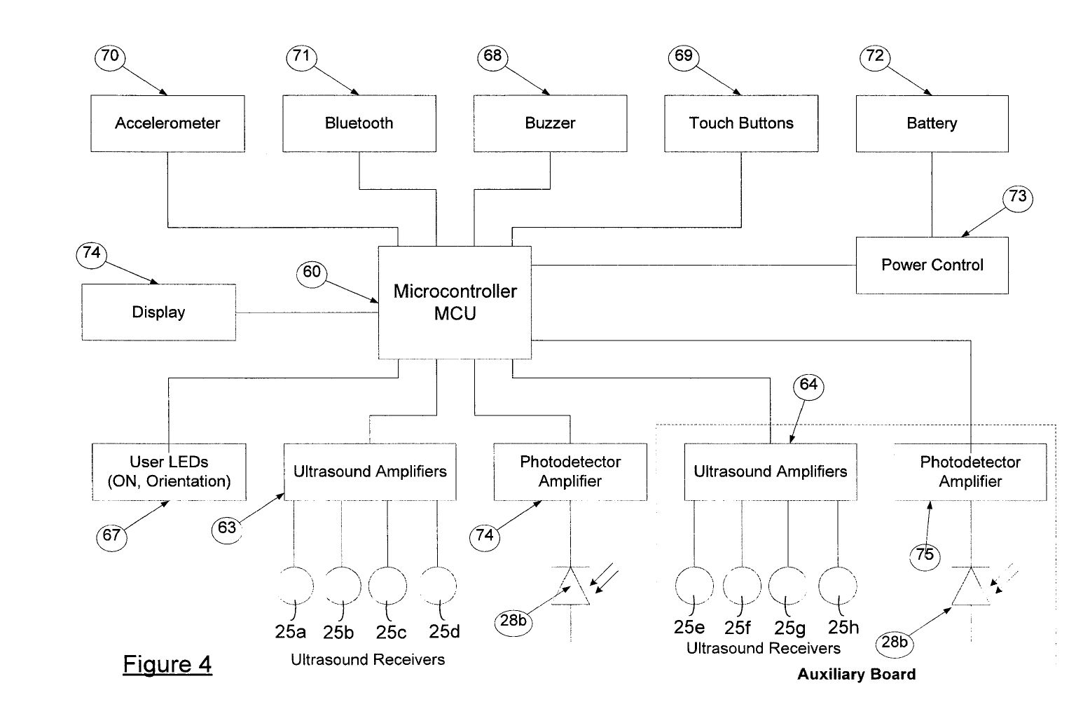

FIG. 4 is a block diagram of an exemplary embodiment of the electronics of a receiver array 24. The unit is controlled by a microcontroller (MCU) 60, which has a number of functional blocks connected to it, each representing hardware and/or software to effect the described function(s) of the block. There are two arrays 24 pointing in different directions in this embodiment, controlled by a single MCU 60. The ultrasound receivers 25a, 25b, 25c, 25d and 25e, 25f, 25g, 25h connect to ultrasound amplifiers 63 and 64, respectively, which amplify and filter the received signal which is sampled by an analog to digital convertor (ADC) integrated in the MCU 60. An external ADC may be used instead, i.e., interposed between each or both of the ultrasound amplifiers 63, 64 and the MCU 60.

Infrared receivers 28a and 28b connect to amplifiers 74 and 75, respectively, which amplify and filter the optical signal for sampling by the ADC. User LEDs 67, the buzzer 68 and touch buttons 69, all of which are coupled to the MCU 60 by conventional electrical coupling means, provide a simple user interface. A three-axis accelerometer 70, also coupled to the MCU 60 by conventional coupling means, provides information on the orientation of the array relative to gravity. The Bluetooth module 71, also coupled to the MCU 60, provides a wireless communication link with the receiver array and the display. A battery 72 and power controller 73, coupled to the MCU 60, provide the required voltages to the units on the array. An optional integrated display 74, coupled to the MCU 60, presents visual information to the user.

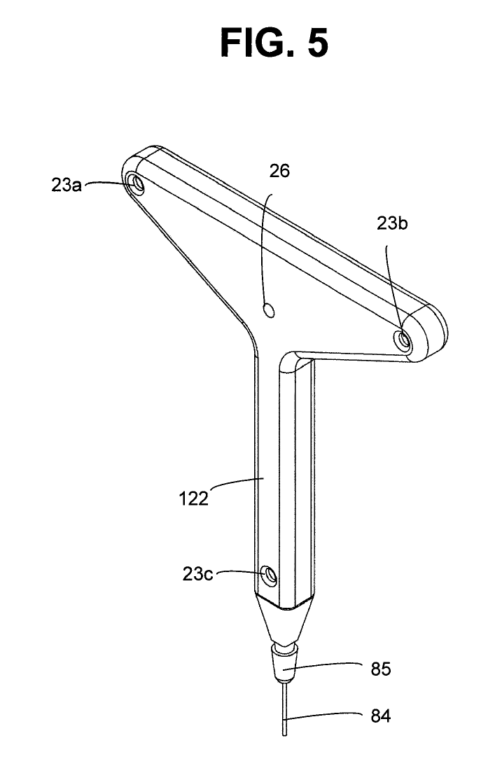

FIG. 5 shows an exemplary embodiment of a transmitter array 22 used as a marker 122. The marker 122 could alternately hold a receiver array 24 which would have the same functionality, as it does not matter for measurement whether the marker 122 is a transmitter or a receiver, or both. In this embodiment, there are three ultrasound transmitters 23a, 23b, 23c, and a single infrared transmitter 26 (see FIGS. 2 and 3). A tip 85 of the marker 12 is designed so that it is a sharp tip for clear indication, and can also accept a hypodermic syringe or needle 84 for piercing the skin for registration on the bone surface (as shown).

The ultrasound transmitters 23a, 23b, 23c are arranged in a triangular shape at known distances from each other, which allows the calculation of the location and orientation of any point on the marker 122 from the 3D locations of each transmitter 23 (see the explanation above). When used as a marker 122 attached to surgical tools, either the same type and shape array can be used, or a differently shaped array can be used. In either case, the functionality is the same.

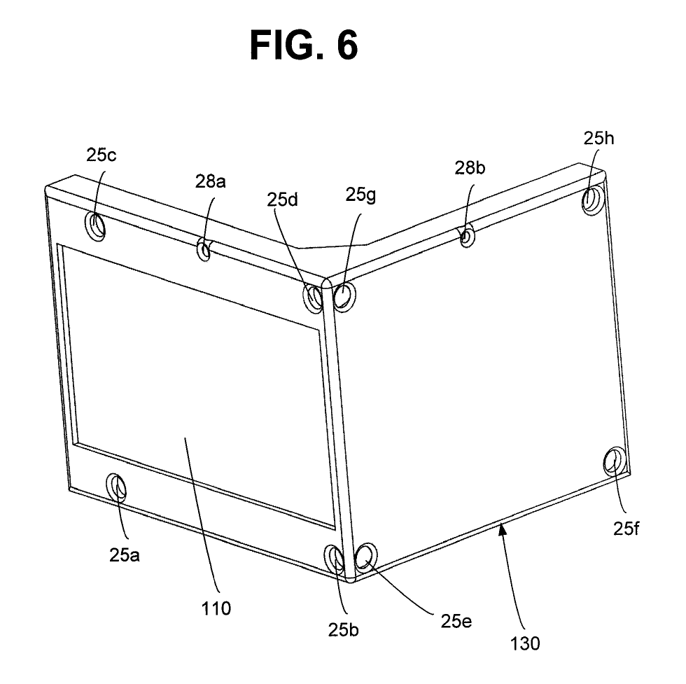

FIG. 6 shows an exemplary embodiment of a reference array 130, with two receiver arrays 24 and a compartment for holding a display 29 (see FIG. 4). This embodiment is typically fixed to a bone, and can be either a receiver or a transmitter, because it does not matter for measurement whether the fixed array is a transmitter or a receiver. There are four ultrasound receivers 25a, 25b, 25c, 25d on one array, and another four ultrasound receivers 25e, 25f, 25g, 25h on the other array which points in a different direction than the first receiver array. There are two optical infrared receivers 28a and 28b, one for each array 24. A cavity 110 holds a display 29, such as an iPod®, which may have a transparent cover and a means to secure the display 29. Not shown is a means to securely attach the fixed array to the bone. Any conventional attachment structure may be used.

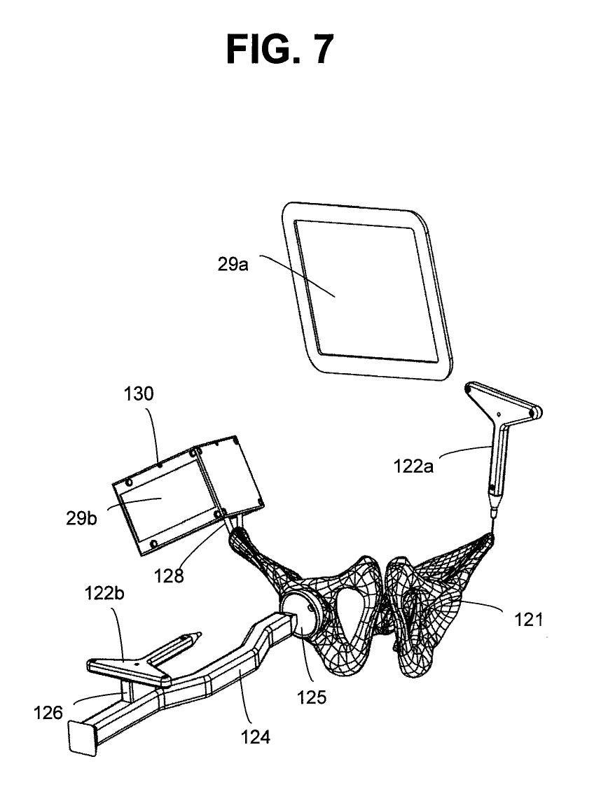

FIG. 7 shows an exemplary embodiment of a complete system as used in a hip replacement operation. Reference array 130, similar to that shown in FIG. 6, is attached to the pelvis 121 by suitable attachment structure, such as screws or pins 128 in the pelvis. There are two markers 122a, 122b: marker 122a functioning as a pointer to indicate anatomical landmarks and marker 122b being attached to a surgical tool 124, e.g., an impacter tool as shown. However, reference number 122 is used to refer to a marker generally. The marker 122a is shown with a hypodermic needle 129 attached, and is pointing to a bony landmark on the pelvis 121. The other marker 122b is connected to the surgical tool 124 by an adapter 126 which is used to position the Acetabular cup 125.

Information obtained by the system may be presented on a large display 29a in the operating room or elsewhere, on a smaller display 29b in cavity 110 on the reference array 130, and/or on integrated display 74 (see FIG. 4) which may be on the receiver array 130 or the transmitter array (not shown in FIG. 7). Reference 29 is used generally to refer a display, of which displays 29a, 29b are two different types.

Adapter 126 may be attached magnetically or by mechanical means, such as a screw socket, to the marker 122b and surgical tool 124. Various adapters 126 may be used to fit the marker 122b to various tools from different manufacturers. A magnetic connection to the adapter 126 allows for quick attachment and removal of the marker 122b from the tool 124.

Generally, the system is implemented as two or more subsystems, where at least one of the subsystems serves as a spatial frame of reference, and the other subsystems can move in space relative to the reference component. The reference subsystem may be referred to as “the reference” and the other movable subsystems as markers. For example in THA, the reference array 130 may be rigidly fixed to the patient's pelvis while the marker 122b could be connected to the (movable) surgical tool 124 such as the tool used to position the acetabular cup 125 (as in FIG. 7 described above). In another example, a reference array 130 could be attached to one leg bone (femur or tibia) while the marker 122b could be attached to the other leg bone on the same leg and to movable surgical tools. Another example is in a TKA operation wherein the reference array 130 is fixed to the operating table or to the ceiling, while markers are connected to the patient's femur and to the tibia, as well as to surgical tools. The reference array 130 need not be fixed in cases where only relative and not absolute spatial relationships are measured.

As described above with reference to FIG. 6, the reference array 130 comprises one or more of arrays of ultrasound components at known distances from each other and at least one optical or electromagnetic component. Both the ultrasound components and the optical components may be transmitters or receivers or bi-directional transceivers. Each component array preferably comprises at least three ultrasound components and at least one optical component. Multiple component arrays may be present on the reference array 130 with each component array pointing in a different direction. One reason for the desirability of orienting the component arrays in different directions is that ultrasound components are not omnidirectional, but have a limited angle for receiving or transmitting energy. To optimize the quality of the received signals, each component array points in a different direction so that signals can be reliably measured from a broad range of angles.

Similarly, the markers 122a, 122b comprise one or more of arrays of ultrasound components on a rigid body at known distances from each other and at least one optical or electromagnetic component. Both the ultrasound components and the optical components may be transmitters, receivers or bi-directional transceivers. Each component array preferably comprises at least three ultrasound components and at least one optical component. However, as the markers 122a, 122b are free to move in space, the typical configuration is that only one array of components is present on each marker.

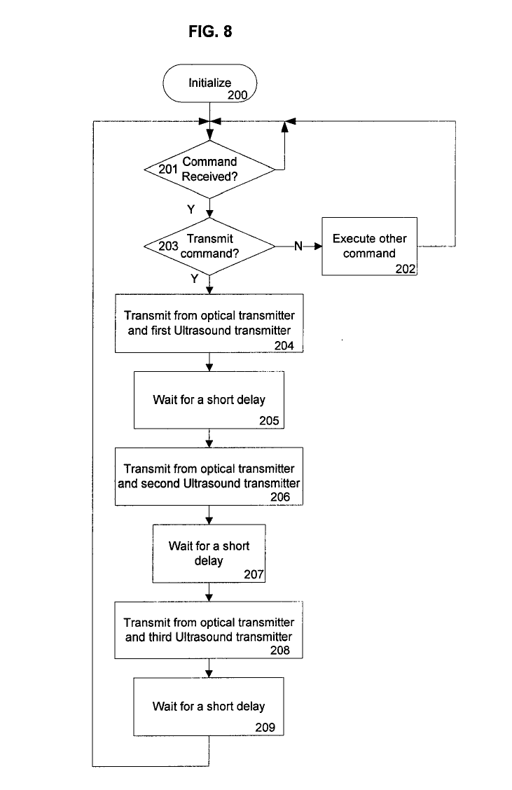

FIG. 8 shows an exemplary embodiment of a flow diagram for the sequence of events typically executed by a transmitter array transmitting a sequence of signals in response to a command from the receiver array. For this sequence, the transmitter array includes an optical transmitter and three ultrasound transmitters. After initialization in step 200, the transmitter array waits for a command in step 201. A determination is made in step 203 whether a transmit command has been received and if the received command received is not a command to transmit a signal, another command is executed in step 202 and then the system returns to step 201 to await another transmission command. However, if a transmit command is received, each transmitter in the transmitter array sequentially sends an optical and ultrasound signal simultaneously, with a short delay between each transmitter's signal. More specifically, in step 204, a first transmitter in the array sends an optical and ultrasound signal simultaneously, followed by a short delay 205. Then, in step 206, a second transmitter sends an optical and ultrasound signal simultaneously, followed by a short delay 207. Then, in step 208, a third transmitter sends an optical and ultrasound signal simultaneously, followed by a short delay 209. The delays 205, 207, 209 may be of equal time or different times. Then, the system returns to step 201 to await another transmission command, or other command.

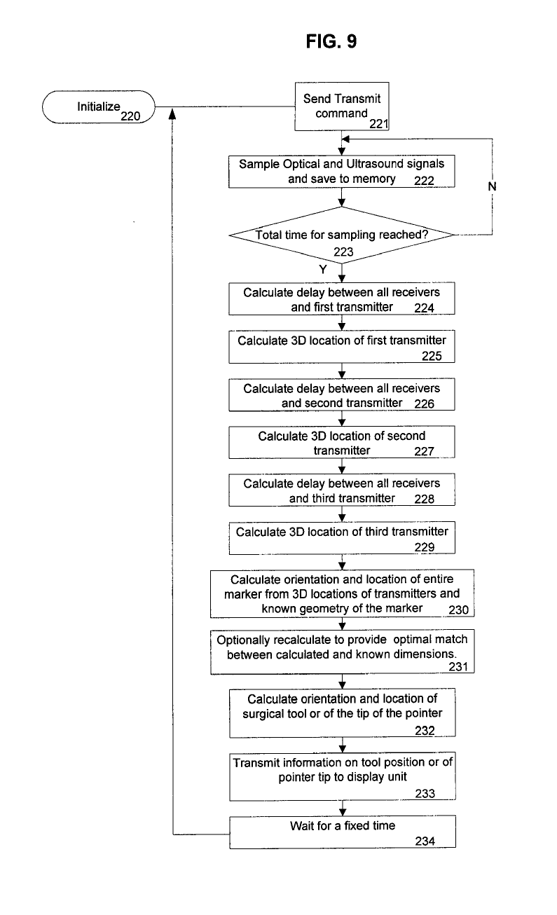

FIG. 9 shows an exemplary embodiment of a flow diagram for the sequence of events typically executed by a receiver array sampling a sequence of signals transmitted in response to a command which it sent. After initialization in step 220, the receiver array sends a command to the transmitter array on a marker 122a, 122b to send a sequence of signals, in step 221. In step 222, the receiver array samples the signals from all of the optical and ultrasound receivers and stores the data in an associated memory. Once enough samples have been acquired so that the maximum delay from the furthest expected transmitter is reached, as determined in step 223, sampling stops and a processor associated with the receiver array begins to calculate the location.

For each receiver channel, the delay is calculated for the first transmitter in step 224. A three-dimensional location of the transmitter is then calculated using the equations above, in step 225. This is repeated for each of the three transmitters sequentially, i.e., the delay is calculated between all of the receivers and the second transmitter in step 226 and a location of the second transmitter is calculated using the equations above in step 227, the delay is calculated between all of the receivers and the third transmitter in step 228 and a location of the third transmitter is calculated using the equations above in step 229.

The orientation and location of the entire marker is then calculated from the determined locations of the transmitters and known geometry of the marker in step 230. Optionally, the orientation and location of the marker is recalculated to provide an optimal match between calculated and known dimensions, in step 231. The calculated dimensions between the transmitters may be compared to the previously determined and known distances and then optionally the locations of the transmitters can be rescaled to provide the best match between calculated and known dimensions.

In step 232, the orientation and location of the surgical tool or a tip of the marker is calculated, and in step 233, may be transmitted to one or more of the displays where the spatial information about this single point at that time is combined with previous samples to provide the information required. After a wait determined by, for example, the update rate in step 234, the procedure returns to step 221 and once again sends a command to the transmitter array to transmit a sequence of signals.

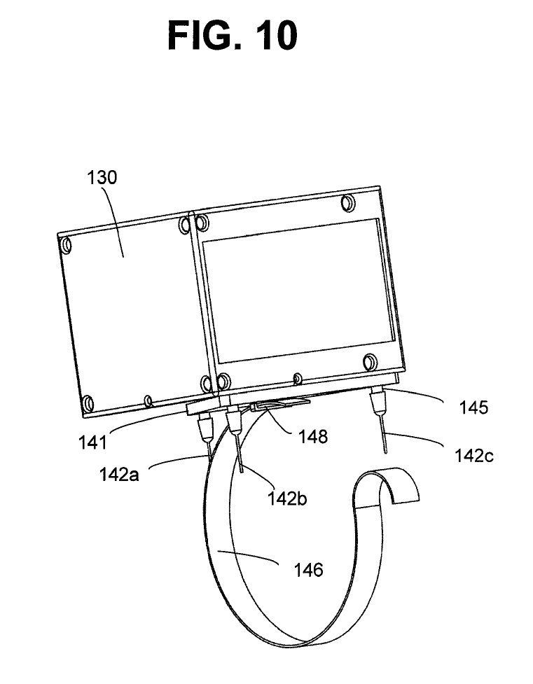

FIG. 10 shows an exemplifying embodiment of a temporary attachment to a patient's body using needles or similar sharp objects pushed against the bone, rather than screwed into the bone. A reference array 130 is attached to a base 141. One or more sharp objects such as needles 142a, 142b and 142c are connected to the base 141, for example using Luer connections 145 or any other suitable means of holding the needles 142a, 142b and 142c. A strap 146 is attached to the base 141, for example, through a loop 148 formed on a side of the base 141 opposite the side on which the reference array 130 is situated.

Strap 146 exemplifies and represents any means for pushing the needles 142a, 142b and 142c against the bone and holding them in place. Strap 146 may be secured to itself, or to the base 141, in any manner known to those skilled in the art. One skilled in the art would understand that multiple variations of this method of attachment are possible, depending on the anatomical structure to which the sharp objects need to be attached, as long as the principle of a temporary attachment to bone using sharp object pushed against the bone is maintained.

Various uses of the structure described above will now be explained.

To make a single measurement of the orientation and location of the marker 122 relative to the reference array 130, the transmitters 23 each sequentially transmit a short ultrasonic signal 14, one after the other. Simultaneously with each ultrasound signal 14 transmission from a transmitter 23, an optical signal 12 with the same temporal characteristics is transmitted (see FIG. 1). For example, if an ultrasound sinusoidal signal of 10 cycles at 40 kHz is transmitted, a square wave optical signal of 10 cycles at 40 kHz is transmitted, with the zero crossings of the sinusoid occurring exactly at the square wave edges. The optical signal 12 and ultrasound signal 14 are amplified at each of the receivers 25 on the receiver array 24 using suitable amplifier circuitry 64 sampled with an analog to digital convertor connected to a microprocessor 60, and converted into digital representations (see FIG. 4). The sampled data for a single position measurement thus comprises three bursts of data, where each burst comprises the signals from three or four ultrasound channels and one optical channel.

Each received ultrasound channel is delayed from the optical channel by a time equal to the distance from the transmitter 23 to that particular receiver 25 divided by the speed of sound. The location of an individual transmitter 23 can then be calculated from the equations set forth above. Measurement of each signal from three transmitters 23a, 23b and 23c is used to calculate the three-dimensional positions of each of the transmitters. As the spatial arrangement of the transmitters 23 on the marker 122 is known, together they allow calculation of the position and orientation of the marker in three-dimensional space relative to the reference 130. This calculation may be performed by microprocessor 60, when provided with, or the ability to access, information about the arrangement of the transmitters 23 on the marker 122.

There may be more than one reference array 130 in use. For example, placing two reference arrays 130 next to the opposite iliac spines creates a geometric configuration with a wider based triangle, such that errors from triangulation to a point at a narrow angle from the reference array 130 are reduced. In addition, it is possible to construct arrays which can take on both transmit and receive functions. For example, using ultrasonic transceivers which can both transmit and receive in the same component, or both transmitter and receiver components on the same rigid body, allow a measurement to be made from one array to the other and then the same measurement to be repeated in the opposite direction. This adds robustness to the system, particularly, in the case of small measurement errors by averaging out the differences between the two measurements.

To track the motion of the marker 122, bursts of signals are transmitted with a repetition rate sufficient to provide a real-time display to the user. For example, at the rates of motion typical of surgical tools, a burst rate of about 5 Hz to about 30 Hz is sufficient to provide smooth updates. The bursts may be transmitted at a constant rate from the marker 122, but as there are likely to be multiple markers 122 and reference arrays 130 in use, controlled transmission is preferred. For example, the reference array 130 can sequentially command each marker 122 to transmit a burst only in response to a wireless command. Unless commanded to do so, the markers 122 do not transmit. The command and control channel can be any wireless or wired communication network such as USB, Zigbee, Bluetooth, an optical communication channel such as IRDA, or an optical or ultrasound signal similar to those implemented on the markers 122 and reference array 130. In the latter case, there is usually a need for an additional receiving element on the transmitter array 21 and an additional transmitting element on the receiver array 24.

The signal format of the synchronized optical signal 12 and ultrasound signal 14 is selected to provide sufficient resolution so that after the mathematical calculations of location, the information is sufficient for the particular application. For example, the acetabular cup 125 (see FIG. 7) need only be located within an angular resolution of about ±10°, while a neurosurgery application might need a precision of well below a millimeter. For lower resolutions, a signal consisting of a few cycles at the ultrasound frequency can provide sufficient resolution. For example at a sampling rate of about 1 megasamples/sec, temporal resolution of the 1 microsecond sampling is equivalent to a spatial resolution of about 344 microns. However, the angular relationships of the reference array 130 and the markers 122 may be that even with this level of spatial resolution for the individual distances, the mathematical manipulations to calculate the position result in a lower resolution than desired.

The transmitter arrays 21, 22 and receiver array 24 may be synchronized with a periodic sync signal. The sync signal may be optical, acoustic or electromagnetic. The transmitters 23 respond to the sync signal by transmitting an optical and acoustic signal after each sync signal, wherein each transmitter 23 transmits at a different time to prevent interference. Each transmitter 23 in the system preferably has a unique identifier which determines when it transmits relative to the sync signal.

A simple method to calculate the delay is to match zero-crossings. The number of cycles and their frequency transmitted is known for both the optical signal 12 and ultrasound signal 14. The delay calculation starts when the software recognizes that an optical signal 12 has been detected and its location in time is calculated by noting the time at which the optical signal 12 crosses the midpoint of its amplitude, which is called a zero-crossing. Similarly, the location in time of the ultrasound signal 14 is calculated from the time at which the ultrasound signal 14 crosses the midpoint of its amplitude. An estimate of the time delay between them is made by a processor that matches the zero-crossings of the optical signal 12 and ultrasound signal 14.

There are a number of methods to improve the resolution, in addition to the improvements above to correct for temperature, humidity and manufacturing tolerances. For example, the sampling rate may be increased to get a higher temporal resolution. The signals may be interpolated to estimate the zero crossing points with a higher precision, than just by comparing to the nearest sampled point. Instead of calculating delay by comparing the delays to zero crossings of the sinusoidal acoustic signal, the cross correlation between the optical signal 12 and ultrasound signal 14 can be calculated, and the peak of the cross-correlation corresponds to the time delay between them.

Due to the characteristics of the transmitter 23 and the drive circuit 42, or in the presence of noise, the rise and fall times of the envelope of the ultrasound signal 14 may not be sharp which leads to uncertainty in the position of the zero crossings relative to the edges of the optical signal 12. In such a case, an alternative signal comprising two tones at different frequencies may be used. This is the well known binary frequency shift keying (BFSK) method of coding. For example, a fixed number of cycles at one frequency (e.g., 40 kHz) followed by a number of cycles at another frequency (e.g., 41 kHz), with this sequence repeated a few times results in an unambiguous signal. The first and last few cycles can be discarded, as the transitions between the two frequencies provide a clear timing point. Cross-correlation of the BFSK signals provides a very high resolution estimate of the time delay between the signals, which is higher than the sample rate.

Use of BFSK also allows each transmitter 23 to be uniquely identified, as each transmitter 23 can transmit a different number of cycles at each frequency. There are multiple alternative formats which can be used besides BFSK, such as a chirp signal or pseudorandom binary sequences, including maximal length sequences which are frequently used to improve signal to noise ratio and are used in GPS.

The information to be presented is displayed on a display 29 for the use by, for example, medical personnel. The display 29 may be either an integral part of either the receiver (see display 52 in FIG. 3) or transmitter (see display 74 in FIG. 4), or may be separate displays 29a and 29b (see FIG. 7). In one embodiment of the invention, the display is attached to a computer which displays the information passed to it via a communication network such as the wireless communication also used for control. A similar alternative is for the display 29 to be part of a tablet computer or a personal media player or any electronic device with computation, display and communication capability. In the example presented in FIG. 7, two exemplary displays are shown, an iPod 29b located in the reference array 130 and an iPad 29a at eye level (with its mounting arrangement partly shown). While there is a benefit to using easily available displays with powerful computation and computation capabilities such as an iPad or iPod (or comparable devices), is clear that any suitable display can be used.

Addition of an accelerometer 70 and 48 to the electronics of the reference array 130 or marker arrays 122 (see FIGS. 3 and 4) provides additional information as to the orientation of the arrays relative to the field of gravity. Knowledge of orientation relative to gravity is not sufficient for surgical navigation, but it does provide an independent means to check that a component or software failure has occurred. By comparing the orientations derived from the acoustic-optical method of the invention, with the measured orientation from the accelerometer, any significant deviation can be flagged as erroneous and the faulty array replaced. The accelerometer also indicates that the marker 122 is being held steady or is being held vertically. Other error checking schemes or techniques are also contemplated to be within the scope and spirit of the invention.

The description here relates at times to a THA operation as an example of how the system is typically used. However, it should be understood that the system may be used in many applications which require surgical navigation or three-dimensional location of medical devices and is not limited to THA operations or procedures. In a THA procedure, the reference array 130 is connected to the patient's pelvis as the first step in using the system during the operation. Alternately, the reference array 130 could be connected to another object in the operating theatre such as the operating table or an overhead arm. In a TKA operation, the reference array 130 could be connected to another bone such as the femur. Pre-operative planning for the required positioning of the implants may be used to define the desired angles and distances of the implants relative to each other and to the patient's bones, using information from CAT, MRI, X-Ray or similar imaging techniques. A member of the medical team then indicates bony landmarks on the pelvis by pointing to them with the tip 85 of a marker 122 (see FIG. 5). The bony landmarks at the two anterior superior iliac spines and at the pubic symphysis or pubic tubercles are frequently used to define a reference plane to which the acetabular cup 125 needs to be aligned at a set angle (see FIG. 7). The plane perpendicular to it and symmetrically between the iliac spines is a second reference plane for the acetabular cup 125.

At each landmark, the location and orientation of the marker 122 is calculated and the position of the tip 85 is calculated from the known position of the tip 85 relative to the ultrasound transmitting-capable components 23. As the bony landmarks may be difficult to precisely locate on some patients, the marker 122 may have a shape at the tip 85 (Luer taper) which allows a hypodermic needle 84 to be attached (see FIG. 7). The marker 122 with the needle 84 is thus pushed against the bone itself, rather than against a layer of fat and skin above the bone. The additional length of the needle 84 is simply added to the dimensions of the marker 122 in the location calculations.

As a next step, the acetabulum is reamed to make a socket for the acetabular cup 125. A marker 122 may be attached to the reamer which is tracked by the reference array 130 and the angle it makes with the pelvis 121 as well as the depth to which it has reamed into the bone are displayed (see FIG. 7). Similarly, a marker on the tool 124 used to place the acetabular cup 125, in this case, an impacter tool, is tracked and the angles it makes with the planes referenced from the bony landmarks are displayed. The surgeon moves and rotates the impacter tool which is used for placing the acetabular cup 125 until the displayed angles are correct and then the acetabular cup 125 is fixed in place. Similar procedures for marking bony landmarks, tracking tools and position of implants are used during the operation to precisely locate the implants according to the surgeons' decisions.

The procedure described above is referred to as “imageless”, as it does not use imaging data of the patient during the operation. In some navigated surgery operations, images from Computed Tomography (CT) or Magnetic Resonance Imaging (MRI) scans or from fluoroscopy are combined with information on the location of tools and implants during the operation. These operations are referred to as “image-guided” operations. The invention described here can be used in both imageless and image-guided operations. As an example, three-dimensional CT data may be displayed on a monitor or display and the physical location of a marker 122 when it is on a landmark or on a fiducial is correlated to the three-dimensional data displayed on the monitor. Registration of a number of anatomical landmarks allows the frame of reference from the reference array 130 to be aligned with the frame of reference of the three-dimensional imaging data. Thus, location of the tools and implants can be viewed according to the actual imaging data from the patient in real time, rather than as calculated parameters derived from pre-operative planning or from generic guidelines.

Use of the system in image guided surgery is not limited to three-dimensional CT or MRI scans but can be integrated with any system which uses imaging, by aligning the frames of reference of the imaging data and the frame of reference of the reference array.

One problem where the reference array 130 is not rigidly attached to the patient's bones is that the patient may move during surgery. For example, if the reference array 130 is attached to the pelvis 121, the motion of the reamer pushing against the pelvis 121 may cause the patient to move, which does not matter if the reference array 130 is attached to the pelvis 121 but does matter if the reference array 130 is on an object in the operating theatre. However, although attaching a reference marker to the patient's bones with surgical pins or screws 128 is a common procedure, it is preferable to avoid the additional trauma to the patient as well as the time associated with attachment.

There are a number of methods to alleviate the problem of patient motion during surgery with a reference array not rigidly connected to the patient's bones with screws or pins 128.
1. In situations where the patient's motion is small, it may be sufficient to hold one or more markers 122 on the patient's body by hand. For example, one or more markers 122 with hypodermic needles 84 may be pushed against the pelvis 121 by hand while the acetabular cup 125 is being positioned. Even though the orientation of the marker 122 may be a little unsteady during the positioning, the tip of the marker 85 or the needle 84 can be held firmly on the pelvis 121 without slipping. The position of the contact points on the pelvis 121 are thus tracked despite the motion of the marker 122 and a calculation of the motion of the pelvis 121 relative to the tool can be made. It may be advantageous for a single marker 122 to have more than one hypodermic needle 84 attached, so that multiple points of contact are established for a single marker.
2. Part of the patient's body may be held immobile during the period of the surgery where navigated procedures are being conducted. This may be achieved by strapping, clamping or otherwise firmly holding part of the patient's body to an immobile object. As an example, a rigid support for the sacral part of the pelvis may be firmly attached to the operating table. A strap, clamp or similar attachment across the front of the patient's pelvis is used to hold the patient's pelvis almost immobile against the sacral support. If the pelvis 121 is firmly held in place, motion of the bony landmarks can be made small enough to allow the operation to still be performed with the angles and distances within the recommended limits. Registration of the reference planes can be done as above, with a reference array 130 affixed to an immobile object such as the ceiling
3. The reference array 130 need not be attached to an immobile object. It may be attached to the patient's body without using surgical screws or pins using straps, clamps or any other non-invasive means, as long as the array is sufficiently stable relative to the patient's body. A rigid support can be strapped or clamped to the patient's body and can move freely along with the patient, but without motion relative to the body part in question. For example, a rigid frame or support on the sacral part of the patient's pelvis with straps or clamps across the front of the pelvis holding it tightly in place, but not attached to the operating table. Placing the straps or clamps so that they push firmly against bone rather than against soft tissue helps ensure that motion is minimized. The reference array 130 is attached to the frame or support and moves with the body part.
4. The reference array 130 can be held in place by hypodermic needles 142a, 142b, 142c pushed firmly against bone, rather than by pins or screws drilled into bone 128 (see FIG. 10). The trauma to the patient is reduced due to smaller wounds and not penetrating into bone. As shown in FIG. 10, the three needles 142a, 142b, 142c form a stable base to which the reference array 130 is connected. An example where this could be used is where a reference array 130 is located on a limb. A strap which goes tightly around the limb pulls the base 141 and the attached needles 142a, 142b, 142c onto the surface of the bone, where the sharp tip prevents the array from slipping. It is clear that the method of reducing trauma to the patient by using needles rather than pins in the bone can also be used to temporarily affix any type of marker used in navigated surgery, such as the active or passive optical arrays used by systems that use cameras and image processing to determine location and orientation.

During imageless navigated hip replacement surgery, it is common to register the location of the edge and the centre of the Acetabulum with the navigation software. This is done by pointing to a number of points along the edge with a marker 122 or by tracing the edge in a semi-continuous circle, and by registering points within the cavity of the Acetabulum. This establishes a reference of the original orientation of the natural Acetabulum and is often the desired orientation of the edge of the implant. However, malalignment may occur if there are errors in the registration due to, for example, surgeon error, limited access through the incision, deformity of the bone or the difficulty of precisely touching the edge.

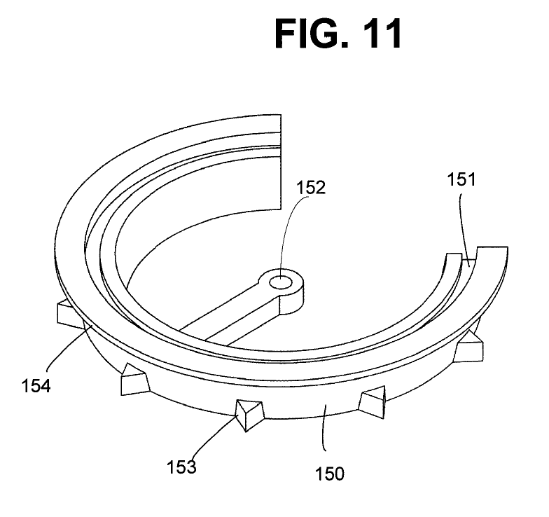

Referring now to FIG. 11, to achieve a quick and precise registration of the edge and the center, a circular shaped insert 150 may be pushed temporarily into place along the edge of, or into the Acetabulum. The insert 150 need not be a complete circle but can be a partial circle or made of subsections so that it is easily inserted and then can snap into place along the edge. A lip 154 or tabs on the outer edge of the insert 150 is designed to contact the edge of the Acetabulum so that the insert 150 does not get pushed into the Acetabulum. The properties of the material of the insert 150 can be selected so that the insert 150 expands into a circular shape once inserted due to the elasticity of the insert 150, or with a mechanism that pushes the insert 150 outwards. The outer peripheral surface of the insert 150 that contacts the bone is roughened or has teeth or barbs 153 so that it grips the bone firmly. A guide 152 aligned with the center of the ring allows the marker 122 to point precisely along the axis through the center of the Acetabulum, and either the marker 122 itself or a needle 84 on the tip of the marker 122 can point to the depth of the natural Acetabulum.

A groove 151 along an upper edge of the insert allows the marker 122 to be traced along the groove 151 and thus indicate the orientation or location of the Acetabulum more precisely. Once the Acetabulum edge and center are registered, the insert 150 is removed to make way for the Acetabular cup 125.

It should be understood that this technique can be used in or on other parts of the body and in other medical procedures with appropriately shaped, temporarily attachable pieces that are aligned to the geometry of the bone. This allows the geometry of the bone to be estimated by registering the location of points on the temporary piece rather than on the bone. For example, one or more protrusions, spines or other anatomical features on a bone can fit into matching depressions on the temporary piece. The location of the protrusions can be determined by touching the marker into a small reference hollow, which is more accessible than touching the protrusions themselves, thus reducing surgeon uncertainty in locating the centre of the protrusion.

An alternative method for determining the position of an anatomical feature is by using two small stereoscopic cameras mounted on the surgical tool 124 itself, e.g., an impacter tool as shown in FIG. 7. The cameras image the anatomical feature and image processing software identifies the salient features, relative to the surgical tool. As an example, in placement of the acetabular cup 125, the edge of the acetabulum is visible to the cameras. Stereoscopic image processing can determine the location and orientation of the acetabular edge within the camera's field of view. As the cameras are rigidly mounted on the surgical tool 124 used to place the acetabular cup 125 at known locations, the angles between the tool and the plane defined by the acetabular edge can be calculated from the stereoscopic image. These angles can then be presented to the surgeon on the display 29.

This approach is also applicable to other anatomical features which can be seen by stereoscopic cameras mounted on any medical device and are preferably spatially aligned to an anatomical feature. The benefit of this method is that it saves time for surgeons as they do not need to manually register the location of the feature with a pointer or marker 122. As time in an operating theatre is expensive, this is a definite benefit. This method is distinctly different from other stereoscopic imaging techniques, which do not image the anatomical feature directly, but image passive or active markers.

Benefits of the present invention over prior art, such as the patents mentioned above, are its low cost and simple implementation, as well as its small size. Acoustic devices such as ultrasound transmitters and receivers are very low cost, well below the cost of stereo cameras. Compared to other location systems based on ultrasound, the invention thus provides an improved method with higher accuracy than systems using only ultrasound. The mathematical processing of the signals by a processor is relatively simple, much less than that required for image processing using cameras, thus enabling simpler and cheaper processors. In addition, all the electronics as well as the display can be contained within the markers and fixed arrays, which does not take any floor space in the operating theatre, while stereo camera based systems are typically mounted on carts which occupy the limited space in the theatre.

Having described exemplary embodiments of the invention with reference to the accompanying drawings, it will be appreciated that the present invention is not limited to those embodiments, and that various changes and modifications can be effected therein by one of ordinary skill in the art without departing from the scope or spirit of the invention as defined by the appended claims. Moreover, although the foregoing invention has been described in some detail by way of illustration and example, for purposes of clarity of understanding, it will be obvious that changes and modifications may be practiced within the scope of the appended claims.

What is claimed is:

1. A method for navigated medical procedures, comprising:
placing at least one transmitter array and at least one receiver array on at least one object used in the medical procedure, each of the at least one transmitter array comprising at least three first transmitters and at least one second transmitter that transmits at a higher speed that the first transmitters, each of the at least one receiver array comprising at least three first receivers that receive transmissions from the first transmitters and at least one second receiver that receives transmissions from the second transmitter;
scheduling a sequence of transmissions by the second transmitter and the at least three first transmitters of the at least one transmitter array to the at least three first receivers and at least one second receiver of the at least one receiver array, wherein in each transmission, the second transmitter and only one of the first transmitters transmit signals and the respective transmitted signals are received by the second receiver and all of the first receivers;
calculating, using a measurement system, distance measurements between each of the first transmitters and each of the first receivers based on time delays between reception of the signals transmitted in each transmission and a speed of sound;
determining a three-dimensional location of at least one of the first transmitters relative to the first receivers from at least three calculated distance measurements between the first transmitter and the first receivers, to thereby provide the three-dimensional location of the at least one object; and
using the three-dimensional location of the at least one object to display information regarding the medical procedure.

2. The method of claim 1, wherein the step of placing the at least one transmitter array and the at least one receiver array on at least one object comprises placing the at least one transmitter array and the at least one receiver array on a common object.

3. The method of claim 1, wherein the step of placing the at least one transmitter array and the at least one receiver array on at least one object comprises placing the at least one transmitter array on a first object and placing the at least one receiver array on a second different object.

4. The method of claim 1, wherein the first transmitters and the first receivers use ultrasound, and the second transmitter and the second receiver use optics.

5. The method of claim 1, wherein the step of placing the at least one transmitter array on the at least one object comprises:
attaching the at least one transmitter array to a rigid body;
fixing the rigid body to the at least one object whereby when the at least one object is a static object or a patient's body, the rigid body constitutes a fixed or reference array, and when the at least one object is a movable object, the rigid body constitutes a marker.

6. The method of claim 1, wherein the step of placing the at least one receiver array on the at least one object comprises:
attaching the at least one receiver array to a rigid body with the at least three first receivers in a non-co-linear arrangement; and
fixing the rigid body to the at least one object whereby when the at least one object is a static object or a patient's body, the rigid body constitutes a fixed or reference array, and when the at least one object is a movable object, the rigid body constitutes a marker.

7. The method of claim 1, wherein the step of placing the at least one transmitter array and the at least one receiver array on the at least one object comprises:
placing the at least one transmitter array on a first object and placing the at least one receiver array on a second different object;
attaching the at least one transmitter array to a first rigid body
attaching the at least one receiver array to a second rigid body with the at least three first receivers in a non-co-linear arrangement;
fixing the first or second rigid body to the first object which is a static object or a patient's body so that the first or second rigid body constitutes a fixed or reference array; and
fixing the other of the first or second rigid body to the second object which is a movable object so that the other of the first or second rigid body constitutes a marker.

8. The method of claim 7, further comprising calculating a three-dimensional location and orientation of one of the transmitter and receiver arrays relative to another of the transmitter or receiver arrays from the location of at least three of the first transmitters and from a known geometry of the transmitter and receiver arrays, whereby a location and orientation of any point on the first and second rigid bodies is calculated from a known geometry of the first and second rigid bodies and the first transmitters and first receivers.

9. The method of claim 1, wherein the receiver array comprises four first receivers, further comprising determining the speed of sound by analyzing signals received by the four receivers from one of the first transmitters.

10. The method of claim 1, further comprising determining the speed of sound by:
analyzing calculated distances between the first transmitters and known dimensions between the first transmitters; or
measuring temperature and optionally humidity in a space through which the signals from the first transmitters travel and adjusting a given speed of sound based on the measured temperature and optionally measured humidity; or
measuring the speed of sound with at least one of the first transmitters and at least one of the first receiver at known distances from each other.

11. The method of claim 1, further comprising:
attaching a hypodermic needle to the at least one transmitter array or the at least one receiver array;
placing the needle into contact with a bony landmark below a surface of the patient's skin, such that a location of the bony landmark is determined from geometry of the at least one transmitter array or the at least one receiver array and the needle.

12. The method of claim 1, further comprising synchronizing the at least one transmitter array and the at least one receiver array with a periodic sync signal.

13. The method of claim 1, further comprising attaching the at least one transmitter array or the at least one receiver array to a rigid body by magnetic force.

14. The method of claim 1, wherein the step of placing the at least one transmitter array and the at least one receiver array on at least one object comprises:
attaching the at least one transmitter array or the at least one receiver array to a rigid body; and
temporarily attaching the rigid body to the patient to thereby constrain relative motion of the rigid body and a part of the patient's body to which the rigid body is temporarily attached.

15. The method of claim 1, wherein the step of placing the at least one transmitter array and the at least one receiver array on at least one object comprises temporarily attaching the at least one transmitter array or the at least one receiver array to a patient by mounting the at least one transmitter or the at least one receiver array on a base and pushing needles attached to the base against a bone of the patient.

16. The method of claim 1, wherein the step of placing the at least one transmitter array and the at least one receiver array on at least one object comprises:
attaching the at least three first transmitters or the at least three first receivers to a rigid body; and
fixing the rigid body to a movable object whereby the rigid body constitutes a marker;
placing a temporary part in or on a bone of a patient, the temporary part having a shape corresponding to at least one anatomical feature of the bone; and
calculating the location or orientation of the at least one anatomical feature of the bone from registering a location of points on the part using the rigid body.

17. The method of claim 16, wherein the part is an insert in the acetabulum and its shape is aligned to the edge of the acetabulum and corresponds to optimal angles at which the acetabular cup is to be placed.

18. A system for navigated medical procedures, comprising:
at least one transmitter array, each of said at least one transmitter array comprising at least three first transmitters and at least one second transmitter that transmits at a higher speed that the first transmitters;
at least one receiver array, each of said at least one receiver array comprising at least three first receivers that receive transmissions from said first transmitters and at least one second receiver that receives transmissions from said second transmitter;
a display system that displays information; and
a measurement system that controls said at least one transmitter array and said at least one receiver array to cause a sequence of transmissions by said second transmitter and all of said first transmitters, wherein in each transmission, said second transmitter and only one of said first transmitters transmit signals and the transmitted signals are received by said second receiver and all of said first receivers;
said measurement system being configured to calculate distance measurements between each of said first transmitters and each of said first receivers based on time delays between reception of the signals transmitted in each transmission and a speed of sound;
said measurement system being further configured to determine a three-dimensional location of at least one of said first transmitters relative to said first receivers from at least three calculated distance measurements between said first transmitter and said first receivers;
whereby placement of said at least one transmitter array or said at least one receiver array on at least one object used in the medical procedure enables the three-dimensional location of the at least one object to be viewed on said display.

19. The system of claim 18, wherein said display is:
mounted on said at least one transmitter array or said at least one receiver array; or
wirelessly coupled to said at least one receiver array.

20. The system of claim 18, wherein said display is separate from said receiver array, further comprising a communications system arranged on said receiver array to communicate with said display.
